(19) United States
(12) Patent Application Publication
(10) Pub. No.: US 2023/0330332 A1
(43) Pub. Date: Oct. 19, 2023

(54) PRESSURE TRANSDUCER FOR INJECTIONS

(71) Applicant: Innovate Our World Consultants, LLC, Houston, TX (US)

(72) Inventor: Ray King, Houston, TX (US)

(21) Appl. No.: 18/042,241

(22) PCT Filed: Aug. 20, 2021
(86) PCT No.: PCT/US2021/046914

§ 371 Date: Feb. 20, 2023

Related U.S. Application Data
(60) Provisional application No. 63/067,875, filed on Aug. 20, 2020.

(51) Int. Cl. A61M 5/168; A61M 5/34

(52) U.S. Cl. CPC A61M 5/16831; A61M 5/344; A61M 5/486

(57) ABSTRACT

The present invention relates generally to methods and systems for ensuring the injection pressure of glue or filler (or other material) for functional and/or cosmetic procedures does not result in retrograde embolus of the injected substance causing functional complication. It does so by indicating the injection pressure, which can be compared vs. the patient's own diastolic blood pressure—by never allowing injection pressure to exceed the diastolic pressure, the occurrence of such embolus into an artery traveling retrograde should be minimized or completely negated. The mechanism of detection of diastolic blood pressure can either be remembered by the user and the injection pressure visually confirmed to remain below this diastolic blood pressure when injecting, or this system may be automated, to ensure that the diastolic blood pressure in a feed-forward mechanism regulates the injection flow pressure when the comparable pressure is neared or reached.

### CROSS-REFERENCE TO RELATED APPLICATIONS

This Patent Cooperation Treaty application claims the benefit and priority of U.S. provisional patent application Ser. No. 63/067,875, filed Aug. 20, 2020, entitled “Pressure Transducer for Functional and/or Cosmetic Injections”, the entire contents of which is hereby incorporated by reference in its entirety.

### FIELD OF THE INVENTION

This invention relates generally to methods and systems for the safe injection of functional and/or cosmetic substance into the vessels of the body, such as to help reduce or obviate the risk of complications (e.g. stroke or blindness) by retrograde injection of the injected substance into another undesired artery.

### BACKGROUND OF THE INVENTION

Present-day injection of a substance for functional and/or cosmetic procedures has a non-negligible risk of retrograde injection, and when injected into an arteriovenous malformation, aneurysm neck, or the face as a filler, can be pushed retrograde into an artery, subsequently filling another artery that then causes an embolism to occlude a key function-supply artery (e.g. causing stroke or compromising vision).

A method that relies upon subjective “slow” injection is oblivious to the actual key force to overcome—the worst-case diastolic blood pressure in a given patient.

### SUMMARY OF THE INVENTION

The present invention relates to methods and systems for accurately measuring the injection pressure of a filler into the arterial vessels of the body for functional and/or cosmetic procedures, and maintaining this injection pressure below a desired pressure. For example, the injection pressure may be kept below a specific patient's diastolic blood pressure, hence reducing or negating the incidence of function-threatening embolism into a subsequent artery due to retrograde substance injection. A pressure transducer, which actively and accurately reflects the pressure of injection, would be useful to reduce or obviate the incidence of retrograde embolus from the injected substance, as by maintaining this injection pressure below the lowest diastolic blood pressure measured in the patient, the risk of such embolism would be negated.

In one aspect of the invention, the pressure transducer would be placed between a syringe and a needle, and an indicator may be utilized that reflects the pressure of the injection target, such as the patient's own diastolic blood pressure, such as through a blood pressure monitor. The desired pressure may then be remembered, recorded or otherwise retained by the user, and used to keep the injection pressure below this value.

In another aspect, the pressure of the injection target, such as the patient's diastolic blood pressure, may be actively measured, and then utilized as an input into a system that measures the injection pressure, and actively alarms, controls or prohibits the injection of substance (e.g. glue or filler) above that number (through a feed-forward mechanism). For example, a computer or other control mechanism may be utilized to control the force, pressure, rate of flow, start/stop and/or other control characteristic applied on the contents of the syringe to produce and/or maintain an injection pressure below the input, and/or to stop injection if the input value is exceeded by the injection pressure. The input may also be set by a user at a desired predetermined level without utilizing active measurement of the pressure of the injection target.

The present invention together with the above and other advantages may best be understood from the following detailed description of the embodiments of the invention illustrated in the drawings.

### BRIEF DESCRIPTION OF THE FIGURES

The drawings accompanying and forming part of this specification are included to depict certain aspects of the invention. A clearer impression of the invention, and of the components and operation of systems provided with the invention, will become more readily apparent by referring to the exemplary, and therefore non-limiting, embodiments illustrated in the drawings, wherein identical reference numerals designate the same components. Note that the features illustrated in the drawings are not necessarily drawn to scale.

FIG. 1 illustrates a schematic view of a syringe and needle with a pressure transducer;

FIG. 1a illustrates a schematic view of a syringe and needle with a pressure transducer with a pressure readout;

FIG. 2 illustrates a schematic view of a syringe and needle with a pressure transducer with a control connection with a control device having an input from a target subject; and

FIG. 3 illustrates a schematic view of a syringe and needle with a pressure transducer with a control connection with a control device having an input from a target subject and a control connection to the actuator of the syringe.

### DETAILED DESCRIPTION OF THE INVENTION

The detailed description set forth below is intended as a description of the presently exemplified methods, devices and compositions provided in accordance with aspects of the present invention, and is not intended to represent the only forms in which the present invention may be practiced or utilized. It is to be understood, however, that the same or equivalent functions and components may be accomplished by different embodiments that are also intended to be encompassed within the spirit and scope of the invention.

Unless defined otherwise, all technical and scientific terms used herein have the same meaning as commonly understood to one of ordinary skill in the art to which this invention belongs. Although any methods, devices and materials similar or equivalent to those described herein can be used in the practice or testing of the invention, the exemplified methods, devices and materials are now described.

The present invention relates to methods and systems for accurately measuring the injection pressure of a filler into the arterial vessels of the body for functional and/or cosmetic procedures, and maintaining this injection pressure below a desired pressure. For example, the injection pressure may be kept below a specific patient's diastolic blood pressure, hence reducing or negating the incidence of function-threatening embolism into a subsequent artery due to retrograde substance injection. A pressure transducer, which actively and accurately reflects the pressure of injection, would be useful to reduce or obviate the incidence of retrograde embolus from the injected substance, as by maintaining this injection pressure below the lowest diastolic blood pressure measured in the patient, the risk of such embolism would be negated. Such embolisms could subsequently travel down another artery branch via which an embolus could compromise key function (e.g. vision) or cause other problems, such as a stroke.

In a typical surgical setting, an injection syringe would be connected to the transducer intervening between the syringe and needle, and reflect the injection pressure; this would be compared to the diastolic blood pressure of a given patient, in an effort to either consciously (per user) control injection pressure to limit it to staying below diastolic pressure, or utilize an automated feed-forward system which indicates such injection pressure approximating or exceeding diastolic blood pressure, or preventing it using an automated mechanism. Other injection devices may generally be utilized in place of a syringe/needle as appropriate or desired in connection with the descriptions below.

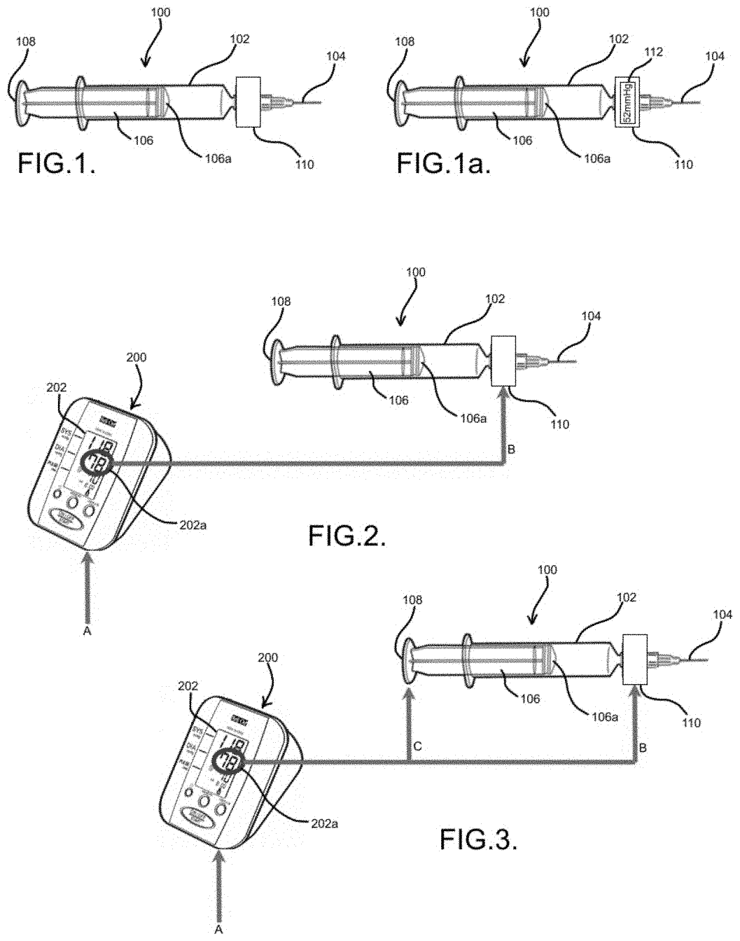

In one aspect of the invention, an embodiment of which is illustrated in the schematic view of FIG. 1, a pressure transducer 110 may be placed between the body 102 of a syringe 100 (which may generally include the reservoir for a substance to be injected) and an injection portion, such as a needle 104, and an indicator may be utilized that reflects the pressure of the injection target, such as the patient's own diastolic blood pressure, such as through a blood pressure monitor or other device. The desired pressure may then be remembered, recorded or otherwise retained by the user, and used to keep the injection pressure below this value, such as by monitoring the measured injection pressure from the pressure transducer 110, as illustrated with the pressure readout 112 in FIG. 1a, and actuating the syringe 100 to apply pressure in the body 102, such as through actuator 108 pushing on or otherwise moving plunger 106 and plunger head 106a. The actuator 108 may be a manual syringe plunger handle or it may also be a mechanical or powered actuator.

The pressure transducer 110 may include, for example, any appropriate form of pressure sensor to detect the injection pressure of the syringe 100, such as a mechanical pressure gauge, MEMS pressure sensor, pressure sensitive valve, strain gauge, piezoelectric sensor, and/or any other appropriate type of sensor.

In another aspect, the pressure of the injection target, such as the patient's diastolic blood pressure, may be actively measured, and then utilized as an input into a system that measures the injection pressure, and actively alarms, controls or prohibits the injection of substance (e.g. glue or filler) above that number (through a feed-forward mechanism). For example, a computer or other control mechanism may be utilized to control the force, pressure, rate of flow, start/stop and/or other control characteristic applied on the contents of the syringe to produce and/or maintain an injection pressure below the input, and/or to stop injection if the input value is exceeded by the injection pressure. The input may also be set by a user at a desired predetermined level without utilizing active measurement of the pressure of the injection target.

FIG. 2 illustrates an embodiment of a syringe 100 with a pressure transducer 110, as similarly to the arrangements in FIGS. 1 and 1a, with a connection B between the pressure transducer 110 and a control device 200. The control device 200 may be, or include, for example, a blood pressure or other target pressure monitor and may include, for example, a display 202 which may show a user the target pressure 202a, and may generally be connected via connection A to the target subject to detect the target pressure, such as through a blood pressure cuff, optical detector or other measuring connection to the target subject. In this arrangement, the detected target pressure, as shown with target pressure 202a, may provide an active input to the pressure transducer 110, which may compare (either itself or via communication with the control device 200) the target pressure 202a and the injection pressure of the syringe 100, and further alert the user (e.g. by an alarm or indicator on the pressure transducer 110 or on the control device 200) or control the injection pressure (e.g. by stopping injection or decreasing pressure) if the injection pressure nears or rises above the target pressure 202a, such that the injection pressure is maintained at or below the target pressure 202a. This may be desirable as the system may then monitor continuously and not rely entirely on the attention, memory and/or manual control of the user.

In some embodiments, such as illustrated in FIG. 3, the control device 200 may also directly act on the syringe 100, as discussed above in regard to control of the injection pressure, such as by, for example, controlling the actuator 108 through connection C. The connection C may, for example, include connection to a motor or other electronically controlled or mechanically controlled actuation mechanism. The control device 200 may also, for example, control actuation through a valve, pressure release or relief mechanism, diverter, or other control mechanism to control the injection pressure, which may be present in, for example, portions of the syringe 100 or as part of the pressure transducer 110.

In some embodiments, the control device 200 may also include computing functionality and/or a connection to a computing device, such as a personal computer, smart phone or other device. The computing functionality or connection may be utilized to control the injection pressure and/or the start/stop of injection.

In some embodiments, the control device 200 may also include a control, such as a start/stop button, which may, for example, enable the start of injection from the syringe 100 and allow manual stop of the injection. The injection may also be automated after start such that the control device 200 controls the actuator 108 in response to the detected injection pressure at the pressure transducer 110 and the measured target pressure 202a.

In some embodiments, the control device 200 may also include functionality for the user to input a desired injection pressure or profile for injection pressure, such as with a predetermined value, a predetermined schedule of values, a function based on the target subject pressure 202a, a function based on the injection pressure detected by the pressure transducer 110, and/or any other appropriate predetermined routine.

Example of Use: Injection Pressure Transduction System for Facial Cosmetic and/or Functional Injection

An injector of substance (e.g. glue or filler) may record the diastolic blood pressure of a given patient, e.g. 80 mm Hg; subsequently this user may keep this in mind, and while injecting using the pressure transducer, never exceed 80 (or even 70) mm Hg, to avoid any chance of retrograde flow, hence minimizing or negating the occurrence of embolus to another artery supplying key anatomical structure for function (e.g. a vessel to the brain, limb, or central retinal artery for vision).

Subsequently, this similar mechanism may involve an automated detection mechanism, such as an arterial line whereby continuous blood pressure readings are available, and supply a diastolic blood pressure which can be coupled to the injection pressure to raise an alarm and/or regulate injection flow (which may also be automated or manually controlled) to be below the diastolic blood pressure.

Reference throughout this specification to “one embodiment”, “an embodiment”, or “a specific embodiment” or similar terminology means that a particular feature, structure, or characteristic described in connection with the embodiment is included in at least one embodiment and may not necessarily be present in all embodiments. Thus, respective appearances of the phrases “in one embodiment”, “in an embodiment”, or “in a specific embodiment” or similar terminology in various places throughout this specification are not necessarily referring to the same embodiment. Furthermore, the particular features, structures, or characteristics of any particular embodiment may be combined in any suitable manner with one or more other embodiments. It is to be understood that other variations and modifications of the embodiments described and illustrated herein are possible in light of the teachings herein and are to be considered as part of the spirit and scope of the invention.

In the description herein, numerous specific details are provided, such as examples of components and/or methods, to provide a thorough understanding of embodiments of the invention. One skilled in the relevant art will recognize, however, that an embodiment may be able to be practiced without one or more of the specific details, or with other apparatus, systems, assemblies, methods, components, materials, parts, and/or the like. In other instances, well-known structures, components, systems, materials, or operations are not specifically shown or described in detail to avoid obscuring aspects of embodiments of the invention. While the invention may be illustrated by using a particular embodiment, this is not and does not limit the invention to any particular embodiment and a person of ordinary skill in the art will recognize that additional embodiments are readily understandable and are a part of this invention.

As used herein, the terms “comprises,” “comprising,” “includes,” “including,” “has,” “having,” or any other variation thereof, are intended to cover a non-exclusive inclusion. For example, a process, product, article, or apparatus that comprises a list of elements is not necessarily limited only those elements but may include other elements not expressly listed or inherent to such process, process, article, or apparatus.

1. A system for controlling injection pressure of an injection device comprising:
an injection device having a substance reservoir and an injection portion;
a pressure transducer disposed between said substance reservoir and said injection portion and adapted to output an injection pressure of said injection device; and
a control device adapted to detect a target subject pressure.

2. The system of claim 1, wherein said injection device comprises a syringe having a plunger adapted to pressurize said substance reservoir to generate said injection pressure and an actuator adapted to control movement of said plunger.

3. The system of claim 1, wherein said injection portion comprises a needle.

4. The system of claim 1, wherein said pressure transducer comprises a pressure sensor selected from the group consisting of a mechanical pressure gauge, MEMS pressure sensor, pressure sensitive valve, strain gauge and a piezoelectric sensor.

5. The system of claim 1, wherein said pressure transducer comprises an output readout adapted to display said injection pressure.

6. The system of claim 1, wherein said control device comprises a blood pressure monitor.

7. The system of claim 1, wherein said control device is adapted to output said target subject pressure.

8. The system of claim 1, wherein said pressure transducer outputs said injection pressure to said control device.

9. The system of claim 8, wherein said control device is adapted to compare said injection pressure and said target subject pressure.

10. The system of claim 1, further comprising a control mechanism adapted to activate when said injection pressure equals or exceeds said target subject pressure.

11. The system of claim 10, wherein said control mechanism is selected from the group consisting of an alarm, alert, valve, pressure release, pressure relief mechanism, diverter and actuator stop.

12. The system of claim 2, wherein said control device is connected to and controls said actuator in response to input from said pressure transducer and said target subject pressure.

13. A method for controlling injection pressure of an injection device comprising:
providing a system comprising:
an injection device having a substance reservoir and an injection portion;
a pressure transducer disposed between said substance reservoir and said injection portion and adapted to output an injection pressure of said injection device; and
a control device adapted to detect a target subject pressure;
measuring said target subject pressure with a pressure sensing device connected to a subject;
injecting a substance from said substance reservoir into a tissue of said subject by pressurizing said substance reservoir with an actuator;
measuring said injection pressure with said pressure transducer; and
controlling said injection pressure via said actuator to maintain said injection pressure at a value equal to or lower than said measured target subject pressure.

14. The method of claim 13, wherein said target subject pressure is a diastolic blood pressure of said subject.

15. The method of claim 13, wherein said substance is a filler or a glue for a cosmetic or functional medical procedure.

16. The method of claim 13, wherein said controlling said injection pressure via said actuator is performed by a user of said system by adjusting movement of said actuator manually in response to said target subject pressure and said output of said injection pressure by said pressure transducer.

17. The method of claim 13, wherein said system alerts a user of said system in response to said injection pressure rising above said target subject pressure.

18. The method of claim 13, wherein said control device maintains said injection pressure below said target subject pressure by controlling said actuator.

19. The method of claim 13, wherein said control device maintains said injection pressure below said target subject pressure via a control mechanism in said pressure transducer.

20. The method of claim 13, wherein said tissue is selected from the group consisting of arteriovenous malformation, aneurysm neck, and face of said subject.
